(19) United States
(12) Patent Application Publication
(10) Pub. No.: US 2025/0349427 A1
(43) Pub. Date: Nov. 13, 2025

(54) METHOD AND APPARATUS FOR GENERATING MEDICAL KNOWLEDGE GRAPH BASED ON TEXT CORPUS

(71) Applicant: Alipay (Hangzhou) Information Technology Co., Ltd., Hangzhou (CN)

(72) Inventors: Xiaodong YAN, Hangzhou (CN); Deng ZHAO, Hangzhou (CN); Zhiqiang ZHANG, Hangzhou (CN); Jinjie GU, Hangzhou (CN); Jun ZHOU, Hangzhou (CN)

(21) Appl. No.: 19/199,398

(22) Filed: May 6, 2025

(30) Foreign Application Priority Data
May 8, 2024 (CN) 202410564986.9

(51) Int. Cl. G16H 50/20; G16H 50/50

(52) U.S. Cl. CPC G16H 50/20; G16H 50/50

(57) ABSTRACT

Embodiments of this specification provide a method and an apparatus for generating a medical knowledge graph based on a text corpus. When a knowledge graph is constructed based on a text corpus, a data obtaining process of the knowledge graph can be divided into two stages: open extraction and alignment. Specifically, entity words and corresponding entity types are first extracted from a raw text corpus in an open manner through a large model, and a corresponding connection relation is further extracted based on the extracted entity words and entity types. Then, entity and relation alignment is performed based on a predefined entity schema and connection schema, and the knowledge graph is constructed based on an alignment result.

### TECHNICAL FIELD

One or more embodiments of this specification relate to the field of computer technologies, and in particular, to a method and an apparatus for generating a medical knowledge graph based on a text corpus.

### BACKGROUND

A knowledge graph is an important branch technology of artificial intelligence, and is a structured semantic knowledge base used to symbolically describe concepts and their relations in the physical world. A basic unit of the knowledge graph is an “entity-relation-entity” triple, and can further correspond to key-value pairs including entities and their related attributes. The entities are connected to each other by using a relation, to form a mesh-like knowledge structure. Knowledge graphs can be classified into a general knowledge graph and a domain-specific knowledge graph based on a function and an application scenario. The general knowledge graph is oriented to general fields, emphasizes the breadth of knowledge, is usually in a form of a structured encyclopedia, and is mainly targeted at ordinary users. The domain-specific knowledge graph is oriented to a specific field, emphasizes the depth of knowledge, usually needs to be constructed based on the database of the industry, and is targeted at practitioners, potential practitioners, etc. in the industry.

With the advent of the large model era, large model-based information extraction (IE) has brought many breakthroughs. With reference to prompt engineering, key information can be extracted from texts in a plurality of fields without preparing supervised samples in advance and training from scratch. This is a great improvement over a conventional extraction model. To construct a knowledge graph, entity words usually need to be extracted and relation information usually needs to be understood from a corpus repository text, that is, two tasks, namely, named entity recognition (NER) and relation extraction (RE), need to be executed. In a conventional technology, a task of extracting knowledge graph entities and connection relation information through a large model is heavy, and attribute description information (schema) usually needs to be provided to the large model in advance, that is, an extracted graph schema is provided in prompt information prompt, and an information extraction result is obtained through question answering (QA). The prompt information in this manner includes the graph schema. When there is a relatively large amount of content in the graph schema, extraction efficiency may be affected. In addition, extracted information is determined based on a predefined schema, which may have limitations. Therefore, how to more effectively extract graph information is an important technical problem in the field of knowledge graph data preprocessing.

### SUMMARY

One or more embodiments of this specification describe a method and an apparatus for generating a knowledge graph based on a text corpus, to resolve one or more problems mentioned in the background.

According to a first aspect, a method for generating a knowledge graph based on a text corpus is provided, including: extracting a plurality of entity words as candidate entity words and entity types to which the candidate entity words belong as candidate entity types from a candidate text corpus through a large model; determining, through the large model by using the plurality of candidate entity words and with reference to the candidate text corpus, a candidate connection relation existing between the plurality of candidate entity words, where the candidate connection relation includes two candidate entities predicted by the large model to have a connection relation and a candidate connection type predicted for the candidate entities; performing an alignment operation on the plurality of candidate entity words, the candidate entity types corresponding to the plurality of candidate entity words, and predefined entity types, to obtain a plurality of target entity types and target entity words in the target entity types; performing an alignment operation on a predefined connection type and the candidate connection type by using the plurality of target entity words, to obtain a target connection relation between the target entity words; and generating knowledge graph data by using the aligned target connection relation.

In an embodiment, extracting the plurality of entity words as the candidate entity words and the entity types to which the candidate entity words belong as the candidate entity types from the candidate text corpus through the large model includes: performing entity word and entity type extraction for a plurality of times through the large model; and obtaining an entity type with a largest quantity of occurrences for a single candidate entity word in an extraction result as a candidate entity type corresponding to the single candidate entity word.

In an embodiment, performing the alignment operation on the plurality of candidate entity words, the candidate entity types corresponding to the plurality of candidate entity words, and predefined entity types includes: obtaining a predefined mapping relation between a candidate entity type and a predefined entity type; and aligning the candidate entity type and the predefined entity type based on the predefined mapping relation.

In a further embodiment, the mapping relation includes: mapping the candidate entity type to the predefined entity type based on at least one of a synonym, a near synonym, and a child class.

In an embodiment, performing the alignment operation on the plurality of candidate entity words, the candidate entity types corresponding to the plurality of candidate entity words, and predefined entity types includes: if a single candidate entity type cannot be aligned with each predefined entity type, outputting at least one of the following selection information items: adding the single candidate entity type as a predefined entity type; mapping the single candidate entity type to a predefined single entity type; and deleting the single candidate entity type; and performing related processing based on a selection result of a user for the selection information items.

In an embodiment, performing the alignment operation on the predefined connection type and the candidate connection type by using the plurality of target entity words, to obtain a target connection relation between the target entity words includes: if a single candidate connection type cannot be aligned with each predefined connection type, outputting the following selection information items: adding the single candidate connection type as a predefined entity type; and deleting the single candidate connection type; and performing related processing based on a selection result of a user for the selection information items.

In an embodiment, generating the knowledge graph data by using the aligned target entity words and target connection relation includes: generating a knowledge graph based on a triple format of a header entity, a connection type, and a tail entity; or generating a knowledge graph based on a quintuple format of a header entity, a connection type, a tail entity, a header entity type, and a tail entity type, where the head entity and the tail entity are described by using target entity words in the target entity types, and the connection type is a target connection type in the aligned target connection relation.

According to a second aspect, an apparatus for generating a knowledge graph based on a text corpus is provided, including:
a first extraction unit, configured to extract a plurality of entity words as candidate entity words and entity types to which the candidate entity words belong as candidate entity types from a candidate text corpus through a large model;
a second extraction unit, configured to determine, through the large model by using the plurality of candidate entity words and with reference to the candidate text corpus, a candidate connection relation existing between the plurality of candidate entity words, where the candidate connection relation includes two candidate entities predicted by the large model to have a connection relation and a connection type predicted for the candidate entities;
a first alignment unit, configured to perform an alignment operation on the plurality of candidate entity words, the candidate entity types corresponding to the plurality of candidate entity words, and predefined entity types, to obtain a plurality of target entity types and target entity words in the target entity types;
a second alignment unit, configured to perform an alignment operation on a predefined connection type and the candidate connection type by using the plurality of target entity words, to obtain a target connection relation between the target entity words; and
a generation unit, configured to generate knowledge graph data by using the aligned target entity words and target connection relation.

According to a third aspect, a computer-readable storage medium is provided. The computer-readable storage medium stores a computer program. When the computer program is executed on a computer, the computer is enabled to perform the method according to the first aspect.

According to a fourth aspect, a computing device is provided, including a memory and a processor. The memory stores executable code, and when the processor executes the executable code, the method according to the first aspect is implemented.

According to the apparatus and the method provided in the embodiments of this specification, when a knowledge graph is constructed based on a text, a data obtaining process of the knowledge graph can be divided into two stages: open extraction and alignment. Specifically, entity words and corresponding entity types are first extracted from a raw text in an open manner through a large model, and a corresponding connection relation is further extracted based on the extracted entity words and entity types. Then, entity and relation alignment is performed based on a predefined entity schema and connection schema, and the knowledge graph is constructed based on an alignment result. In this way, entity types and entity words can be more comprehensively mined, and when prompt information is reduced, efficiency of mining the entity word and the connection relation by the large model is improved. Therefore, comprehensiveness and effectiveness of the constructed knowledge graph can be improved.

### BRIEF DESCRIPTION OF DRAWINGS

To describe the technical solutions in the embodiments of this specification more clearly, the following briefly describes the accompanying drawings needed for describing the embodiments. Clearly, the accompanying drawings in the following description show merely some embodiments of this specification, and a person of ordinary skill in the art can derive other drawings from these accompanying drawings without creative efforts.

FIG. 1 is a schematic diagram of a specific implementation architecture for mining knowledge graph data based on a text in a conventional technology;

FIG. 2 is a schematic diagram of a specific implementation architecture for generating knowledge graph data based on a text in the technical concept of this specification;

FIG. 3 is a schematic diagram of a procedure of generating a knowledge graph based on a text corpus according to an embodiment of this specification;

FIG. 4 is a schematic diagram of a specific example of extracting an entity word and a connection relation according to an embodiment of this specification;

FIG. 5 is a schematic diagram of a specific example of entity type and connection type alignment according to an embodiment of this specification;

FIG. 6 is a schematic diagram of a procedure framework for generating a knowledge graph based on a text corpus according to a specific example of the technical concept of this specification; and

FIG. 7 is a structural block diagram of an apparatus for generating a knowledge graph based on a text corpus according to an embodiment of this specification.

### DESCRIPTION OF EMBODIMENTS

The solutions provided in this specification are described below with reference to the accompanying drawings.

Two important tasks in graph construction are named entity recognition (NER) and relation extraction (RE). Entities/Relations in a specific field and entity types/relation types corresponding to the entities/relations are extracted by using raw texts (referred to as text corpora below) such as sentences and paragraphs (paragraph) in documents. Quality of a finally constructed knowledge graph is directly determined based on effects of the two tasks. NER and RE are two subfields of an information extraction task. Large language model (LLM, which can also be referred to as a large model in this specification)-based information extraction has promoted intelligent LLM-based graph construction.

Usually, input data of the large model can include a task description and a processing object, and output data is determined by using the processing object. In some implementations, the input data of the large model can further include prompt information. The prompt information can include an example, description information of the input data and/or the output data, etc.

In an existing general large model-based information extraction framework, for example, ChatIE and DeepKE-LLM, a plurality of information extraction tasks can be unified in a same framework. However, an extracted graph schema needs to be provided in a prompt, and an information extraction result is obtained through QA. The graph schema can include an entity schema and a connection schema. The entity schema can be used to describe at least one of an entity word, a type to which an entity belongs, entity description information, etc. The connection schema can be used to describe at least one of a connection type between entities, connection relation description information, etc.

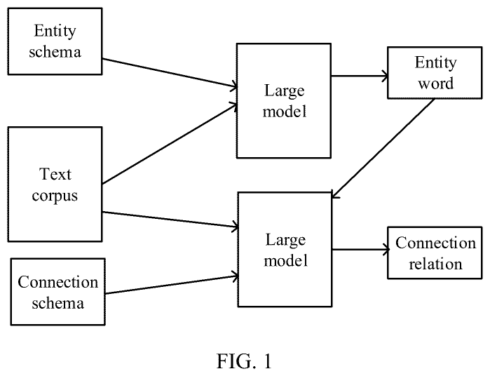

As shown in FIG. 1, a knowledge graph data mining framework in a conventional technology is provided. In this framework, first, an entity schema is input together with a text corpus into a large model, and the large model extracts a corresponding entity word from the text corpus by using the entity schema as prompt information. Then, the entity word and a connection schema are input as prompt information together with the text corpus into the large model, so that the large model extracts a connection relation between entities by using the text corpus.

According to the knowledge graph data extraction method in this framework, the large model may forcibly map some unrelated entities to an entity type defined in the entity schema, and extract a relatively large quantity of invalid entities. For a graph with a large quantity of entity types and connection types, if all graph schema information is placed in the prompt information prompt, an input prompt information length (for example, a token size) of the LLM may be exceeded. Consequently, the graph schema information cannot be entirely placed at a time, affecting effectiveness of information extraction.

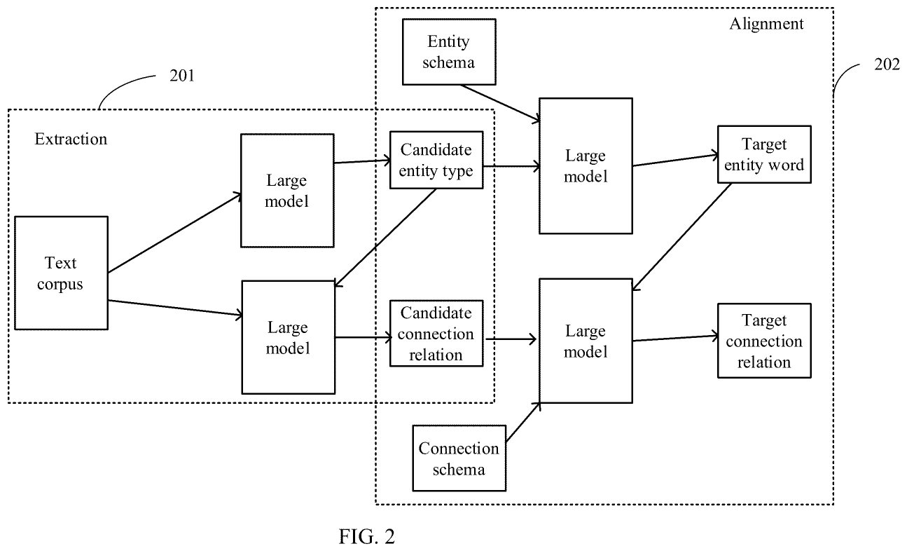

In view of this, a new graph data extraction framework is used in this specification, and a graph data extraction process is divided into two stages: information extraction and alignment. Specifically, an entity word and a connection relation are first extracted from a text corpus in an open manner, and then an entity type and the connection relation are sequentially aligned by using a graph schema. FIG. 2 is a schematic diagram of a specific implementation architecture of this specification. In FIG. 2, first, a schematic block 201 shows an information extraction process. In a process of extracting an entity word by using a large model, an entity schema may not be used as prompt information, that is, an entity word in a related field is extracted without a limitation (that is, in an open manner), to obtain several candidate entity words, and a candidate entity type corresponding to each candidate entity word is determined. In addition, both the candidate entity word and a text corpus are used as processing objects of the large model, and when a connection schema is not used as prompt information, the large model extracts a connection relation, recorded as a candidate connection relation, between the candidate entity words based on the text corpus. Then, with reference to a schematic block 202, a candidate entity type is used as a processing object, and the large model performs entity word alignment by using an entity type in a predefined entity schema as prompt information, to obtain a target entity type and a target entity word in each target entity type. The alignment process can be a process of performing at least one of mapping to a same or similar entity type and addition of an entity type. In addition, the target entity word and the candidate connection relation are used as processing objects, a connection type in a predefined connection schema is used as prompt information, and the large model performs connection relation alignment to obtain a target connection relation. When the target entity word and a target connection type are fused, a connection relation identified by a triple (header entity, relation type, tail entity) or a connection relation of a quintuple (header entity, relation type, tail entity, header entity type, tail entity type) can be obtained. The triple or the quintuple can constitute a basic unit of a knowledge graph.

In this architecture, the information extraction process is performed through open extraction, and is not limited by predefined graph schema information. Therefore, a problem of excessively long prompt information can be avoided, and a possible entity type and a possible connection type can be recalled to a maximum extent. In this way, a more accurate and complete knowledge graph can be constructed for related service processing, to improve service processing effectiveness.

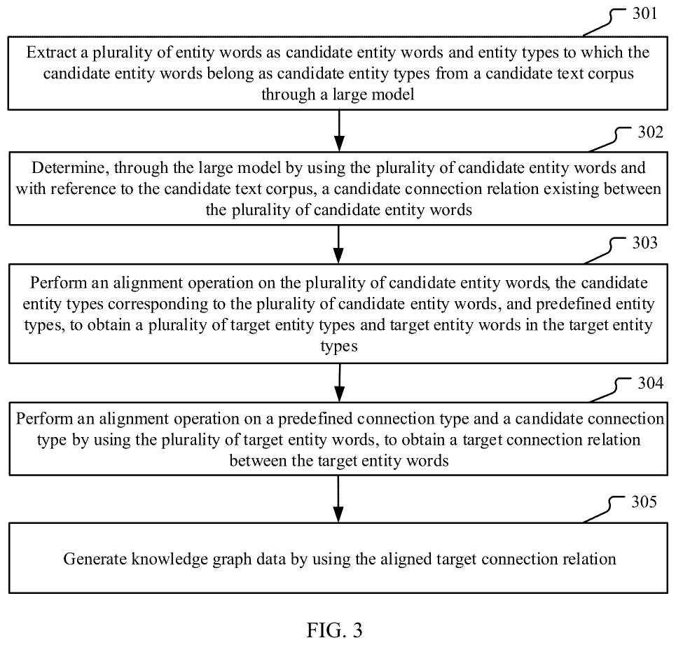

The following describes the technical concept of this specification in detail with reference to an embodiment shown in FIG. 3.

FIG. 3 is a schematic diagram of a procedure of generating a knowledge graph based on a text corpus according to an embodiment. The procedure can be performed by a computer, a device, or a server that has a specific computing capability. As shown in FIG. 3, the procedure of generating a knowledge graph based on a text provided in this embodiment of this specification can include the following steps: Step 301: Extract a plurality of entity words as candidate entity words and entity types to which the candidate entity words belong as candidate entity types from a candidate text corpus through a large model. Step 302: Determine, through the large model by using the plurality of candidate entity words and with reference to the candidate text corpus, a candidate connection relation existing between the plurality of candidate entity words, where the candidate connection relation includes two candidate entities predicted by the large model to have a connection relation and a connection type predicted for the candidate entities. Step 303: Perform an alignment operation on the plurality of candidate entity words, the candidate entity types corresponding to the plurality of candidate entity words, and predefined entity types, to obtain a plurality of target entity types and target entity words in the target entity types. Step 304: Perform an alignment operation on a predefined connection type and the candidate connection type by using the plurality of target entity words, to obtain a target connection relation between the target entity words. Step 305: Generate knowledge graph data by using the aligned target connection relation.

First, in step 301, a plurality of entity words are extracted as candidate entity words and entity types to which the candidate entity words belong are extracted as candidate entity types from the candidate text corpus through the large model.

The text corpus can be a corpus in a corpus repository. Here, the corpus repository can include corpus information obtained in advance from a plurality of channels. For example, the obtaining channel is a professional book, a related field network platform, a paper, etc. In an example of a medical field knowledge graph, the corpus repository can include knowledge retrieved from a network, medical-related books, etc. The network here can include a medical-related website, for example, ** search, an encyclopedia medical-related term, and another related web resource obtained through web crawling.

A single text corpus can be a sentence, a paragraph, etc. In some embodiments, for network knowledge, a text chunking operation can also be performed. Text chunking is a process of splitting a large text chunk into small chunks, namely, a process of obtaining a small chunk of text corpus. A single small chunk can be used as a text corpus. When content is embedded by using the LLM, text chunking can be used to optimize accuracy of content recalled from a vector database. In addition, the medical-related books can be various books on medical pathology and medical advice and medicine seeking, for example, Compendium of Materia Medica and other specialized books. In an optional embodiment, for professional data, a text tree (for example, recorded as a DOM tree) can be generated by using an image recognition technology (for example, OCR), and a paragraph library is formed based on parsing of the text tree. A single paragraph in the paragraph library is a single text corpus.

It can be understood that an entity word is a named entity of an entity, and represents the corresponding entity. In the technical concept of this specification, a process of extracting an entity word from a text corpus can be an open process, that is, no limitation is constituted by using a predefined entity schema as prompt information, and only a description of an extraction task is provided to the large model.

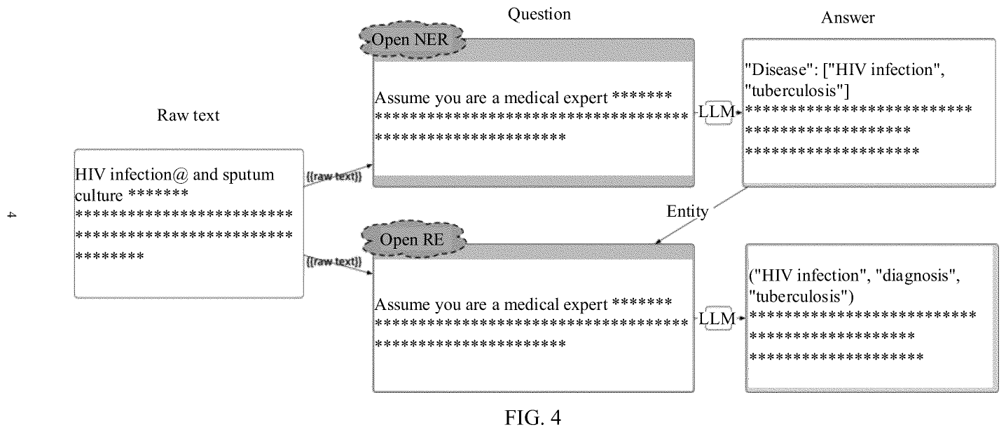

In a specific example, when a medical knowledge graph is constructed, as shown in FIG. 4, task description information (Question) can be input into the large model: “Assume you are a medical expert and you are provided with a text. Please find all possible entities and types corresponding to the entities from the text and subject information. The answer format is Dict: {‘entity type’: [text, text], . . . }, and the provided processing object is text—{{raw text}}. The raw text points to a text corpus named “raw text” on a left side of FIG. 4: “There is HIV infection@ and sputum culture positive for Mycobacterium tuberculosis, and the result of the chest X-ray examination is consistent with tuberculosis manifestations. Therefore, tuberculosis is diagnosed. The current medications for HIV infection@ include anti-tuberculosis drugs and pyridoxine.” In this way, the large model can process the raw text based on the task description, extract a plurality of medical entity words from the raw text, and summarize corresponding entity types.

As shown in FIG. 4, the extracted entity words “HIV infection” and “tuberculosis” are classified into an entity type “disease”; the extracted entity words “sputum culture” and “chest X-ray examination” are classified into an entity type “examination”; and so on. The extracted entity words are recorded, for example, as an entity list “entity”.

It can be learned that the large model can extract several candidate entity words based on a provided text corpus and task description information extracted based on an entity word, and automatically classify the candidate entity words into different candidate entity types.

In practice, there may be noise interference in some text corpora. For example, for the text corpus “Before DMARD treatment for rheumatoid arthritis@ is started, examinations for hepatitis B, hepatitis C, purified protein derivative (PPD), complete blood count, and liver function need to be performed . . . ”, entity words “hepatitis B” and “hepatitis C” may be recognized. Due to interference of context noise such as “examinations need to be performed”, the entity words may be recognized as an entity type “examination”. Therefore, in an optional implementation, to reduce the context noise interference as much as possible, when a candidate entity word is extracted, entity word extraction can be performed on a same text corpus for a plurality of times, and an entity word and an entity type with a largest quantity of occurrences in an extraction result can be obtained as the candidate entity word and a corresponding candidate entity type. For example, extraction is performed for three times, and the obtained entity word “hepatitis B” corresponds to the entity type “disease” for two times, and corresponds to the entity type “examination” for one time. In this case, the entity type “disease” with a largest quantity of occurrences is selected as a candidate entity type corresponding to the candidate entity word “hepatitis B”.

Then, in step 302, a candidate connection relation existing between the plurality of candidate entity words is determined through the large model by using the plurality of candidate entity words and with reference to the candidate text corpus.

The candidate connection relation can be used to describe two candidate entities predicted by the large model to have a connection relation and a connection type predicted for the candidate entities. Further, the connection type can be used to describe a connection type between two entities. For example, a connection type between entity types “disease” and “examination item” can be “examination”. In the technical concept of this specification, the connection relation can also be extracted through open extraction. That is, instead of providing a prompt by using a predefined connection type, the large model predicts a possible connection relation between the plurality of candidate entity words extracted in step 301 based on the text corpus in the corpus repository, and predicts a connection relation type, namely, a candidate connection type, of the connection relation.

When the candidate connection type is extracted, the candidate entity words and the corresponding entity types can be input into the large model, and the large model detects a possible connection relation between the candidate entity words based on semantic recognition of the corpus. In a specific example shown in FIG. 4, task description information (Question) can be input into the large model: “Assume you are a medical expert and you are provided with a text of a paragraph of a document and a list of entities included in the paragraph content. Please determine a relation between these entities. The answer format is a list: {(header entity, relation, tail entity), . . . }, and the provided processing objects are text—{{raw text}} and entity List—{{entity}}. The following connection relations can be obtained by calling the large model: (“HIV infection ”, “diagnosis”, “tuberculosis”), (“tuberculosis”, “examination method”, “sputum culture”), (“tuberculosis”, “examination method”, “chest X-ray examination”), (“sputum culture”, “examination result”, “positive for Mycobacterium tuberculosis”), etc.

It can be learned that in a connection relation prediction process, the large model can predict a connection relation between entities of different entity types, and can further predict a connection relation between entities of a same entity type. “Diagnosis”, “examination method”, “examination result”, etc. are used as connection types predicted for corresponding two entities. The connection relation predicted by the large model needs to be further processed, and therefore is referred to as the candidate connection relation here.

Then, in step 303, an alignment operation is performed on the plurality of candidate entity words, the candidate entity types corresponding to the plurality of candidate entity words, and predefined entity types, to obtain a plurality of target entity types and target entity words in the target entity types.

Here, both the candidate entity word and the candidate entity type are extracted by the large model in an open manner based on the task description information, and are not limited by the predefined entity type. The extracted candidate entity word, candidate entity type, etc. may be the same as the predefined entity type or different from the predefined entity type. For example, the extracted candidate entity type may be a new entity type, may be a near-synonymous expression of the predefined entity type, or may be a child class or a parent class of the predefined entity type. Therefore, the extracted candidate entity type can be calibrated and aligned by using the predefined entity type, that is, an alignment operation (calibration and alignment can be performed) with the predefined entity type is performed on the plurality of candidate entity types.

The alignment operation between the entity types can be performed manually, can be performed through a pre-trained semantic model based on semantic matching, or can be performed through the large model. This is not limited here.

When alignment is performed through the pre-trained semantic model based on semantic matching, the candidate entity type and the predefined entity type can be separately embedded based on semantic recognition, to obtain corresponding semantic representation vectors, and then the candidate entity type can be mapped to the predefined entity type based on a similarity between the semantic representation vector of the candidate entity type and the semantic representation vector of the predefined entity type.

When alignment is performed through the large model, optionally, a predefined mapping relation can be added as reference information, and the predefined mapping relation can be used to map some synonyms, near synonyms, and child class words to predefined entity types. For example, the candidate entity type “examination” is mapped to the entity type “examination method”, the candidate entity type “sign” is mapped to the entity type “symptom”, the candidate entity type “operation” is mapped to the entity type “treatment”, and so on.

According to a possible design, for a candidate entity type that cannot be aligned when alignment is performed based on the semantic matching model or the large model, alignment can be manually performed, or an alignment suggestion is provided by the model. For example, an alignment suggestion is provided to include at least one of the following selection information items: adding a single candidate entity type that cannot be aligned as a predefined entity type; mapping the single candidate entity type that cannot be aligned to a predefined single entity type; and deleting the single candidate entity type that cannot be aligned. In this case, a candidate information item can be manually selected, and the execution body performs related processing based on selection of a user.

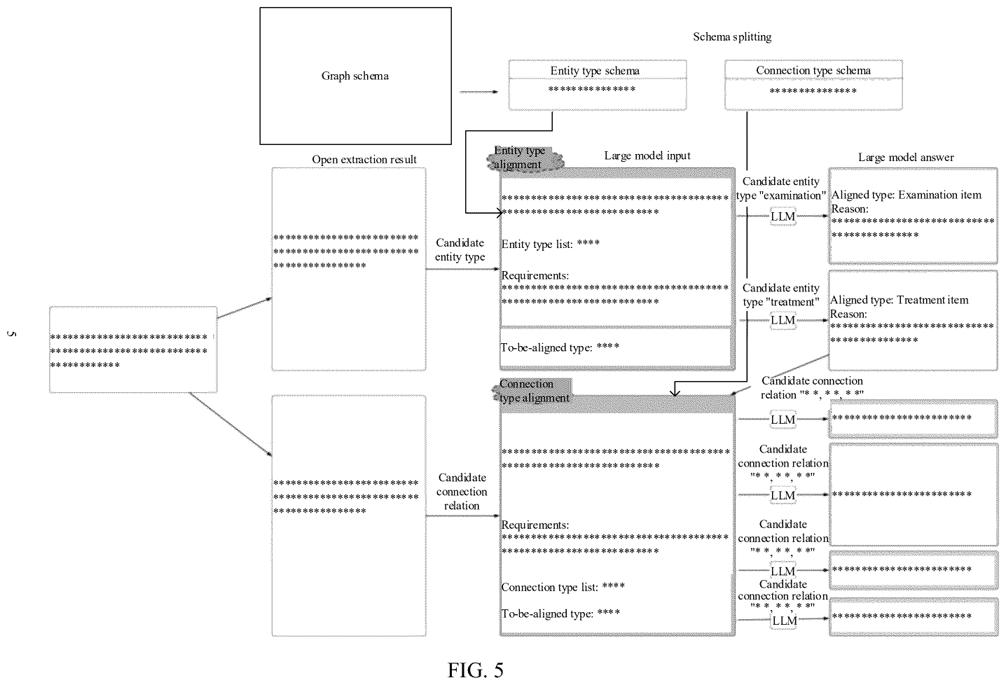

In an example, in FIG. 5, when entity type alignment is performed through the large model, task description information can be provided: “There is currently a task of entity type alignment in a knowledge graph, with the purpose of aligning a to-be-aligned type with a predefined entity type list. Please find an entity type most similar to the to-be-aligned type from the entity type list, and provide an answer with “Aligned entity type: XX”. Otherwise, output “No aligned entity type”. In addition, a predefined entity type list such as (source_type) and a to-be-aligned type list (including the candidate entity type) such as (entity_type_schema) are provided. Optionally, other requirements can be further imposed. For example, the candidate entity type can be mapped to the parent class and cannot be mapped to the child class, and a reason for alignment or non-alignment is provided. In this way, the large model can align the candidate entity type in the to-be-aligned type list with the entity type in the predefined entity type list, and give a reason. For example, the reasons why the candidate entity type “examination” can be aligned with the entity type “examination item”, and the candidate entity type “treatment” can be aligned with the entity type “treatment item” are provided and are that the alignment requirements in the task are met.

In an embodiment not shown, the large model may provide a candidate entity type that cannot be aligned and a reason for non-alignment. For example, the candidate entity type “medical” is a parent class of all entity types in the predefined entity type list and cannot aligned with any entity type. In this case, the large model can output a reason for non-alignment, and can provide an option of whether to use the candidate entity type “medical” as a new entity type or delete the candidate entity type “medical”. After manual selection, a corresponding operation is performed based on a manual confirmation result.

In this way, each candidate entity type can be aligned with the predefined entity type, but is not limited to the predefined entity type, so that more comprehensive entity types are mined. After the candidate entity type is aligned with the predefined entity type, an aligned entity type can be recorded as a target entity type. An entity word corresponding to the target entity type is referred to as a target entity word.

Further, in step 304, an alignment operation is performed on a predefined connection type and the candidate connection type by using the plurality of target entity words, to obtain a target connection relation between the target entity words.

The predefined connection type can be, for example, a connection relation type defined in a connection schema, and can be used as a prior knowledge to correct the candidate connection type. Here, the candidate connection type is extracted by the large model in an open manner based on the task description information, and is not limited by the predefined connection type. Therefore, the extracted connection type, etc. may be the same as the predefined connection type or different from the predefined connection type. For example, the extracted connection type may be a new relation type, may be a near-synonymous expression of the predefined connection type, or may be a child class or a parent class of the predefined connection type. Similar to the candidate entity type, the extracted candidate connection type can be calibrated and aligned by using the predefined connection type, that is, an alignment operation (calibration and alignment can be performed) with the predefined connection type is performed on the plurality of candidate connection types.

Similarly, connection type alignment can be performed manually, or can be performed through the large model. This is not limited here. When alignment is performed through the large model, a predefined mapping rule can be added as reference information, and the predefined mapping rule can be used to map some near synonyms, child classes, and parent-class connection types to predefined connection types.

It can be understood that during connection type alignment, not only alignment between semantic expressions of the connection types is considered, but also target entity types of two connected entities is involved. Therefore, the target entity type can be used as reference information for performing connection relation alignment by the large model.

According to a possible design, for a candidate connection type that cannot be aligned when alignment is performed based on the semantic matching model or the large model, alignment can be manually performed, or an alignment suggestion is provided by the model to manually determine whether to perform alignment based on the processing suggestion. For example, the processing suggestion here can include at least one of the following: adding a single candidate connection type that cannot be aligned as a predefined connection type; forcibly mapping the single candidate connection type that cannot be aligned to a predefined single connection type; and deleting the single candidate connection type that cannot be aligned. The large model can output a reason for non-alignment and a processing suggestion, and it is manually chosen to delete the connection relation in the processing suggestion.

In an optional implementation, connection relation alignment includes alignment between the connection types, and further includes alignment between entity types corresponding to two entity words connected to the connection type. For example, if a target entity type of a tail entity is “disease” and a connection relation is “diagnosis”, a target entity type of a header entity can only be “examination result” or “symptom”. Otherwise, no alignment can be performed, and the connection relation cannot hold. For example, in a candidate connection relation, target entity types corresponding to both two aligned entities are “disease”, the candidate connection relation is “diagnosis”, and a connection relation is “disease, diagnosis, and disease”, which is clearly improper. In this case, the large model can output a reason for non-alignment and a processing suggestion, and it is manually chosen to delete the connection relation in the processing suggestion.

After the candidate connection type is aligned with the predefined connection type, a connection relation such as (entity word in the target entity type, connection type, entity word in the target entity type) can be obtained, and can be recorded as a target connection relation here.

Then, in step 305, knowledge graph data is generated by using the aligned target connection relation.

It can be understood that the target entity type is a result obtained by performing alignment with the predefined entity type, and the target connection relation describes a connection between entity words in the target entity type based on the target connection type. Therefore, a knowledge graph can be generated based on entities connected to the target connection relation.

In an embodiment, the target connection relation can be described as a triple (header entity, connection type, tail entity). A knowledge graph can be generated by using each triple.

For example, each triple is recorded as a knowledge graph, or a visual knowledge graph is constructed by using triple information.

In another embodiment, the predefined entity type and other related information (for example, a source, a collection time, etc.) can be used as schema information of a corresponding entity, to form a quintuple (header entity, connection type, tail entity, header entity type, tail entity type), and a knowledge graph is generated based on the generated quintuple. The process of generating the knowledge graph based on the quintuple is similar to the process of generating the knowledge graph based on the triple. When a visual knowledge graph is constructed, (header entity, connection type, tail entity) can be used as basic composition of the knowledge graph, and the header entity type and the tail entity type are respectively used as attribute information (schema information) of the header entity and the tail entity, to form the knowledge graph.

In the knowledge graph constructed in this way, entity types corresponding to the header entity and the tail entity are not limited to an entity type predefined in the entity schema, and connection types can be not limited to a connection type predefined in the relation schema. In addition, when an entity word is mined, there is no need to input the entity schema in advance, to avoid a problem that a running speed is slow and efficiency is relatively low because of excessively long prompt information of the entity schema.

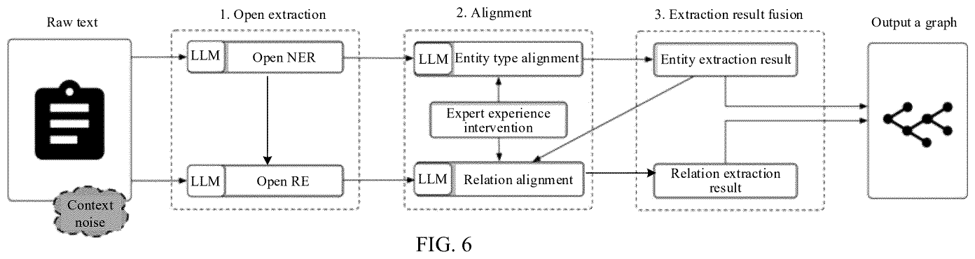

To further clarify the technical concept of this specification, FIG. 6 is a schematic diagram of a knowledge graph construction procedure according to a specific example of the technical concept of this specification. As shown in FIG. 6, a raw text is first obtained. The raw text is allowed to include context noise. Then, at an open extraction stage, entity word extraction (NER) and connection relation extraction (RE) are sequentially performed through a large model. Further, at an alignment stage, expert experience can be introduced in both an entity alignment process and a relation alignment process. Entity types are aligned to obtain an entity extraction result that includes a target entity type. In addition, relation types are aligned based on the expert experience and the entity extraction result, to obtain a relation extraction result that includes a target relation type. Then, at an extraction result fusion stage, a knowledge graph is output based on the entity extraction result and the relation extraction result.

The above-mentioned process is reviewed. According to the method for generating a knowledge graph based on a text corpus provided in the technical concept of this specification, when a knowledge graph is constructed based on a text corpus, a data obtaining process of the knowledge graph can be divided into two stages: open extraction and alignment. Specifically, entity words and corresponding entity types are first extracted from a raw text in an open manner through a large model, and a corresponding connection relation is further extracted based on the extracted entity words and entity types. Then, entity and relation alignment is performed based on a predefined entity schema and connection relation schema, and the knowledge graph is constructed based on an alignment result. In this way, entity types and entity words can be more comprehensively mined, and when prompt information is reduced, efficiency of mining the entity word and the connection relation by the large model is improved. Therefore, comprehensiveness and effectiveness of the constructed knowledge graph can be improved.

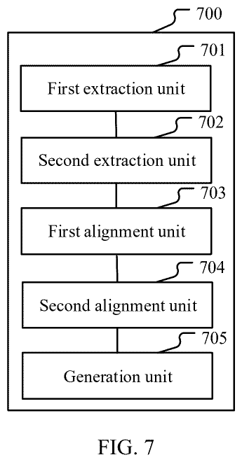

According to an embodiment of another aspect, an apparatus for generating a knowledge graph based on a text corpus is further provided. The apparatus can be disposed in a computer, a terminal, or a server having a specific computing capability. FIG. 7 shows an apparatus 700 for generating a knowledge graph based on a text according to an embodiment. As shown in FIG. 7, the apparatus 700 can include:
a first extraction unit 701, configured to extract a plurality of entity words as candidate entity words and entity types to which the candidate entity words belong as candidate entity types from a candidate text corpus through a large model;
a second extraction unit 702, configured to determine, through the large model by using the plurality of candidate entity words and with reference to the candidate text corpus, a candidate connection relation existing between the plurality of candidate entity words, where the candidate connection relation includes two candidate entities predicted by the large model to have a connection relation and a connection type predicted for the candidate entities;
a first alignment unit 703, configured to perform an alignment operation on the plurality of candidate entity words, the candidate entity types corresponding to the plurality of candidate entity words, and predefined entity types, to obtain a plurality of target entity types and target entity words in the target entity types;
a second alignment unit 704, configured to perform an alignment operation on a predefined connection type and the candidate connection type by using the plurality of target entity words, to obtain a target connection relation between the target entity words; and
a generation unit 705, configured to generate knowledge graph data by using the aligned target entity words and target connection relation.

In an embodiment, the first extraction unit 701 can be further configured to: perform entity word and entity type extraction for a plurality of times through the large model; and obtain an entity type with a largest quantity of occurrences for a single candidate entity word in an extraction result as a candidate entity type corresponding to the single candidate entity word.

In an embodiment, the first alignment unit 703 can be further configured to: obtain a predefined mapping relation between a candidate entity type and a predefined entity type; and align the candidate entity type and the predefined entity type based on the predefined mapping relation.

The mapping relation can include: mapping the candidate entity type to the predefined entity type based on at least one of a synonym, a near synonym, and a child class.

In an embodiment, the first alignment unit 703 can be further configured to: if a single candidate entity type cannot be aligned with each predefined entity type, output at least one of the following selection information items: adding the single candidate entity type as a predefined entity type; mapping the single candidate entity type to a predefined single entity type; and deleting the single candidate entity type; and perform related processing based on a selection result of a user for the selection information items.

In an embodiment, the second alignment unit 704 can be further configured to: if a single candidate connection type cannot be aligned with each predefined connection type, output the following selection information items: adding the single candidate connection type as a predefined entity type; and deleting the single candidate connection type; and perform related processing based on a selection result of a user for the selection information items.

In an embodiment, the generation unit 705 can be configured to: generate a knowledge graph based on a triple format of a header entity, a connection type, and a tail entity; or generate a knowledge graph based on a quintuple format of a header entity, a connection type, a tail entity, a header entity type, and a tail entity type.

The head entity and the tail entity are described by using target entity words in the target entity types, and the connection type is a target connection type in the aligned target connection relation.

It should be noted that the apparatus 700 shown in FIG. 7 corresponds to the method described in FIG. 3, and corresponding descriptions in the method embodiment shown in FIG. 3 are also applicable to the apparatus 700. Details are omitted here for simplicity.

According to an embodiment of another aspect, a computer-readable storage medium is further provided. The computer-readable storage medium stores a computer program, and when the computer program is executed on a computer, the computer is enabled to perform the method described with reference to FIG. 3, etc.

According to an embodiment of still another aspect, a computing device is further provided, and includes a memory and a processor. The memory stores executable code, and when the processor executes the executable code, the method described with reference to FIG. 3, etc. is implemented. A person skilled in the art should be aware that in the above-mentioned one or more examples, the functions described in the embodiments of this specification can be implemented by hardware, software, firmware, or any combination thereof. When this specification is implemented by software, the functions can be stored in a computer-readable medium or transmitted as one or more instructions or code in a computer-readable medium.

The objectives, technical solutions, and beneficial effects of the technical concept of this specification are further described in detail in the above-mentioned specific implementations. It should be understood that the above-mentioned descriptions are merely specific implementations of the technical concept of this specification, but are not intended to limit the protection scope of the technical concept of this specification. Any modification, equivalent replacement, improvement, etc. made based on the technical solutions of the embodiments of this specification shall fall within the protection scope of the technical concept of this specification.

1. A method for generating a knowledge graph based on a text corpus, comprising:
extracting a plurality of entity words as candidate entity words and entity types to which the candidate entity words belong as candidate entity types from a candidate text corpus through a large model;
determining, through the large model by using the plurality of candidate entity words and with reference to the candidate text corpus, a candidate connection relation existing between the plurality of candidate entity words, wherein the candidate connection relation comprises two candidate entities predicted by the large model to have a connection relation and a candidate connection type predicted for the candidate entities;
performing an alignment operation on the plurality of candidate entity words, the candidate entity types corresponding to the plurality of candidate entity words, and predefined entity types, to obtain a plurality of target entity types and target entity words in the target entity types;
performing an alignment operation on a predefined connection type and the candidate connection type by using the plurality of target entity words, to obtain a target connection relation between the target entity words; and
generating knowledge graph data by using the aligned target connection relation.

2. The method according to claim 1, wherein extracting the plurality of entity words as the candidate entity words and the entity types to which the candidate entity words belong as the candidate entity types from the candidate text corpus through the large model comprises:
performing entity word and entity type extraction for a plurality of times through the large model; and
obtaining an entity type with a largest quantity of occurrences for a single candidate entity word in an extraction result as a candidate entity type corresponding to the single candidate entity word.

3. The method according to claim 1, wherein performing the alignment operation on the plurality of candidate entity words, the candidate entity types corresponding to the plurality of candidate entity words, and predefined entity types comprises:
obtaining a predefined mapping relation between a candidate entity type and a predefined entity type; and
aligning the candidate entity type and the predefined entity type based on the predefined mapping relation.

4. The method according to claim 3, wherein the mapping relation comprises: mapping the candidate entity type to the predefined entity type based on at least one of a synonym, a near synonym, and a child class.

5. The method according to claim 1, wherein performing the alignment operation on the plurality of candidate entity words, the candidate entity types corresponding to the plurality of candidate entity words, and predefined entity types comprises:
if a single candidate entity type cannot be aligned with each predefined entity type, outputting at least one of the following selection information items: adding the single candidate entity type as a predefined entity type; mapping the single candidate entity type to a predefined single entity type; and deleting the single candidate entity type; and
performing related processing based on a selection result of a user for the selection information items.

6. The method according to claim 1, wherein performing the alignment operation on the predefined connection type and the candidate connection type by using the plurality of target entity words, to obtain a target connection relation between the target entity words comprises:
if a single candidate connection type cannot be aligned with each predefined connection type, outputting the following selection information items: adding the single candidate connection type as a predefined entity type; and deleting the single candidate connection type; and
performing related processing based on a selection result of a user for the selection information items.

7. The method according to claim 1, wherein generating the knowledge graph data by using the aligned target entity words and target connection relation comprises:
generating a knowledge graph based on a triple format of a header entity, a connection type, and a tail entity; or
generating a knowledge graph based on a quintuple format of a header entity, a connection type, a tail entity, a header entity type, and a tail entity type,
wherein the head entity and the tail entity are described by using target entity words in the target entity types, and the connection type is a target connection type in the aligned target connection relation.

8. A non-transitory computer-readable storage medium, wherein the non-transitory computer-readable storage medium stores a computer program, which when executed by a processor causes the processor to:
extract a plurality of entity words as candidate entity words and entity types to which the candidate entity words belong as candidate entity types from a candidate text corpus through a large model;
determine, through the large model by using the plurality of candidate entity words and with reference to the candidate text corpus, a candidate connection relation existing between the plurality of candidate entity words, wherein the candidate connection relation comprises two candidate entities predicted by the large model to have a connection relation and a candidate connection type predicted for the candidate entities;
perform an alignment operation on the plurality of candidate entity words, the candidate entity types corresponding to the plurality of candidate entity words, and predefined entity types, to obtain a plurality of target entity types and target entity words in the target entity types;
perform an alignment operation on a predefined connection type and the candidate connection type by using the plurality of target entity words, to obtain a target connection relation between the target entity words; and
generate knowledge graph data by using the aligned target connection relation.

9. The non-transitory computer-readable storage medium according to claim 8, wherein the processor being caused to extract the plurality of entity words as the candidate entity words and the entity types to which the candidate entity words belong as the candidate entity types from the candidate text corpus through the large model comprises being caused to:
perform entity word and entity type extraction for a plurality of times through the large model; and
obtain an entity type with a largest quantity of occurrences for a single candidate entity word in an extraction result as a candidate entity type corresponding to the single candidate entity word.

10. The non-transitory computer-readable storage medium according to claim 8, wherein the processor being caused to perform the alignment operation on the plurality of candidate entity words, the candidate entity types corresponding to the plurality of candidate entity words, and predefined entity types comprises being caused to:
obtain a predefined mapping relation between a candidate entity type and a predefined entity type; and
align the candidate entity type and the predefined entity type based on the predefined mapping relation.

11. The non-transitory computer-readable storage medium according to claim 10, wherein the mapping relation comprises being caused to: map the candidate entity type to the predefined entity type based on at least one of a synonym, a near synonym, and a child class.

12. The non-transitory computer-readable storage medium according to claim 8, wherein the processor being caused to perform the alignment operation on the plurality of candidate entity words, the candidate entity types corresponding to the plurality of candidate entity words, and predefined entity types comprises being caused to:
if a single candidate entity type cannot be aligned with each predefined entity type, output at least one of the following selection information items: adding the single candidate entity type as a predefined entity type; mapping the single candidate entity type to a predefined single entity type; and deleting the single candidate entity type; and
perform related processing based on a selection result of a user for the selection information items.

13. The non-transitory computer-readable storage medium according to claim 8, wherein the processor being caused to perform the alignment operation on the predefined connection type and the candidate connection type by using the plurality of target entity words, to obtain a target connection relation between the target entity words comprises being caused to:
if a single candidate connection type cannot be aligned with each predefined connection type, output the following selection information items: adding the single candidate connection type as a predefined entity type; and deleting the single candidate connection type; and
perform related processing based on a selection result of a user for the selection information items.

14. A computing device, comprising a memory and a processor, wherein the memory stores executable code, and when the processor executes the executable code, the computing device is caused to:
extract a plurality of entity words as candidate entity words and entity types to which the candidate entity words belong as candidate entity types from a candidate text corpus through a large model;
determine, through the large model by using the plurality of candidate entity words and with reference to the candidate text corpus, a candidate connection relation existing between the plurality of candidate entity words, wherein the candidate connection relation comprises two candidate entities predicted by the large model to have a connection relation and a candidate connection type predicted for the candidate entities;
perform an alignment operation on the plurality of candidate entity words, the candidate entity types corresponding to the plurality of candidate entity words, and predefined entity types, to obtain a plurality of target entity types and target entity words in the target entity types;
perform an alignment operation on a predefined connection type and the candidate connection type by using the plurality of target entity words, to obtain a target connection relation between the target entity words; and
generate knowledge graph data by using the aligned target connection relation.

15. The computing device according to claim 14, wherein the computing device being caused to extract the plurality of entity words as the candidate entity words and the entity types to which the candidate entity words belong as the candidate entity types from the candidate text corpus through the large model comprises being caused to:
perform entity word and entity type extraction for a plurality of times through the large model; and
obtain an entity type with a largest quantity of occurrences for a single candidate entity word in an extraction result as a candidate entity type corresponding to the single candidate entity word.

16. The computing device according to claim 14, wherein the computing device being caused to perform the alignment operation on the plurality of candidate entity words, the candidate entity types corresponding to the plurality of candidate entity words, and predefined entity types comprises being caused to:
obtain a predefined mapping relation between a candidate entity type and a predefined entity type; and
align the candidate entity type and the predefined entity type based on the predefined mapping relation.

17. The computing device according to claim 16, wherein the mapping relation comprises being caused to: map the candidate entity type to the predefined entity type based on at least one of a synonym, a near synonym, and a child class.

18. The computing device according to claim 14, wherein the computing device being caused to perform the alignment operation on the plurality of candidate entity words, the candidate entity types corresponding to the plurality of candidate entity words, and predefined entity types comprises being caused to:
if a single candidate entity type cannot be aligned with each predefined entity type, output at least one of the following selection information items: adding the single candidate entity type as a predefined entity type; mapping the single candidate entity type to a predefined single entity type; and deleting the single candidate entity type; and
perform related processing based on a selection result of a user for the selection information items.

19. The computing device according to claim 14, wherein the computing device being caused to perform the alignment operation on the predefined connection type and the candidate connection type by using the plurality of target entity words, to obtain a target connection relation between the target entity words comprises being caused to:
if a single candidate connection type cannot be aligned with each predefined connection type, output the following selection information items: adding the single candidate connection type as a predefined entity type; and deleting the single candidate connection type; and
perform related processing based on a selection result of a user for the selection information items.

20. The computing device according to claim 14, wherein the computing device being caused to generate the knowledge graph data by using the aligned target entity words and target connection relation comprises being caused to:
generate a knowledge graph based on a triple format of a header entity, a connection type, and a tail entity, or
generate a knowledge graph based on a quintuple format of a header entity, a connection type, a tail entity, a header entity type, and a tail entity type,
wherein the head entity and the tail entity are described by using target entity words in the target entity types, and the connection type is a target connection type in the aligned target connection relation.
